(19) United States
(12) Patent Application Publication
(10) Pub. No.: US 2017/0164846 A1
(43) Pub. Date: Jun. 15, 2017

(54) PRESSURE SENSING GUIDEWIRE SYSTEMS INCLUDING AN OPTICAL CONNECTOR CABLE

(71) Applicant: BOSTON SCIENTIFIC SCIMED, INC., MAPLE GROVE, MN (US)

(72) Inventor: LLOYD RADMAN, BLAINE, MN (US)

(73) Assignee: BOSTON SCIENTIFIC SCIMED, INC., MAPLE GROVE, MN (US)

(21) Appl. No.: 15/372,380

(22) Filed: Dec. 7, 2016

Related U.S. Application Data
(60) Provisional application No. 62/266,230, filed on Dec. 11, 2015.

(51) Int. Cl. A61B 5/0215; F16B 1/00; A61B 5/02; A61B 5/00

(52) U.S. Cl. CPC A61B 5/02154; A61B 5/6851; F16B 1/00; A61B 5/02028; A61B 2562/228; A61B 2562/223; F16B 2001/0035

(57) ABSTRACT

An optical connector cable assembly including a first portion and a second portion, wherein the first portion is magnetically couplable to the second portion. A first optical fiber is disposed within the optical connector cable assembly extending from the first portion. The second portion is configured to connect to a guidewire including an optical pressure sensor and a second optical fiber extending proximally from the optical pressure sensor.

### CROSS-REFERENCE TO RELATED APPLICATIONS

This application claims the benefit of priority under 35 U.S.C. §119 to U.S. Provisional Application . No. 62/266,230, filed Dec. 11, 2015, the entirety of which is incorporated herein by reference.

### TECHNICAL FIELD

The present disclosure pertains to medical devices, and methods for manufacturing medical devices. More particularly, the present disclosure pertains to blood pressure sensing guidewires.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. In one aspect, the disclosure relates to an optical connector cable assembly, the optical connector cable assembly including a first portion and a second portion, wherein the first portion is magnetically couplable to the second portion. A first optical fiber is included with the optical connector cable assembly extending from the first portion. The second portion is configured to connect to a guidewire including an optical pressure sensor and a second optical fiber extending proximally from the optical pressure sensor.

Alternatively or additionally to any of the embodiments above, the first portion and the second portion are rotatably and magnetically couplable

Alternatively or additionally to any of the embodiments above, the second portion is coupled to a rotatable connector.

Alternatively or additionally to any of the embodiments above, the rotatable connector is a rotatable collet.

Alternatively or additionally to any of the embodiments above, the optical connector cable assembly is configured to connect to a signal processing module.

Alternatively or additionally to any of the embodiments above, a magnetic force rotatably holds the first connector cable and the second connector cable together via a magnet and a magnetic receptive material.

Alternatively or additionally to any of the embodiments above, the first portion comprises a female adapter and the second portion comprises a male adapter.

Alternatively or additionally to any of the embodiments above, the female adapter is coupled to a pressure sensing guidewire.

Alternatively or additionally to any of the embodiments above, the first portion comprises a magnet having a first polarity and the second portion comprises a magnet having a second polarity that is opposite to that of the first polarity.

In another aspect, the disclosure relates to a medical device system for measuring blood pressure, the system including an optical connector cable assembly including a first portion and a second portion magnetically coupled to the first portion, the first portion comprising an optical connector cable including a first optical fiber. A pressure sensing guidewire includes a pressure sensor and a second optical fiber extending proximally from the pressure sensor, the first optical fiber is capable of optically communicating the second optical fiber. The second portion of the optical connector cable assembly is coupled to the guidewire.

Alternatively or additionally to any of the embodiments above, the first portion and the second portion are rotatably and magnetically couplable.

Alternatively or additionally to any of the embodiments above, the second portion includes a rotatable proximal connector capable of being rotatably coupled to the guidewire.

Alternatively or additionally to any of the embodiments above, the rotatable proximal connector is a rotatable collet.

Alternatively or additionally to any of the embodiments above, the pressure sensing guidewire comprises a tubular member having a proximal region and a distal region, the second optical fiber is disposed within the distal region of the tubular member, the second optical fiber is coupled to the optical pressure sensor, the optical pressure sensor is disposed within the tubular member along the distal region.

Alternatively or additionally to any of the embodiments above, a centering member is coupled to an outer surface of the first optical fiber and positioned adjacent to the optical pressure sensor, the centering member configured to reduce contact between an inner surface of the tubular member and the optical pressure sensor.

Alternatively or additionally to any of the embodiments above, the centering member is coupled to an inner surface of the distal region of the tubular member.

Alternatively or additionally to any of the embodiments above, the optical connector cable assembly is configured to connect to a signal processing module.

Alternatively or additionally to any of the embodiments above, the first portion comprises a first magnet having a first polarity and the second portion comprises a second magnet having a second polarity opposite to that of the first polarity.

In another aspect, the disclosure relates to a method of connecting a pressure sensing guidewire to an optical connector cable. The method includes advancing a first portion of an optical connector cable assembly into contact with a second portion of the optical connector cable assembly such that the first portion and second portion are magnetically held together and a first optical fiber of the optical connector cable assembly and a second optical fiber are in optical communication therein. The first optical fiber is disposed within the optical cable assembly and the second optical fiber extends from a pressure sensing guidewire having a pressure sensor. The pressure sensing guidewire is coupled to the second portion of the optical connector cable assembly.

Alternatively or additionally to any of the embodiments above, the first portion and the second portion are rotatably and magnetically coupled.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional side view of a portion of an example medical device;

FIG. 2 is a partial cross-sectional view of an example medical device disposed at a first position adjacent to an intravascular occlusion;

FIG. 3 is a partial cross-sectional view of an example medical device disposed at a second position adjacent to an intravascular occlusion;

FIG. 4 is an exploded partial cross-sectional view of an example of a medical is device system;

FIG. 5 is a side view of an example medical device system;

FIG. 6 is a side view of an example medical device system with parts connected;

FIG. 7 is an enlarged view taken at section 7 in FIG. 6;

FIG. 8 is a partial cross-sectional view of another example of a medical device system before connection;

FIG. 9 is a partial cross-sectional view of a medical device system similar to that shown in FIG. 8 after connection;

FIG. 10 is a partial cross-sectional view of another example of a medical device system with parts connected; and FIG. 11 is a partial cross-sectional view of another example of a medical device system similar to that shown in FIG. 10 in an alternative configuration with parts connected.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

During some medical interventions, it may be desirable to measure and/or monitor the blood pressure within a blood vessel. For example, some medical devices may include pressure sensors that allow a clinician to monitor blood pressure. Such devices may be useful in determining fractional flow reserve (FFR), which may be understood as the ratio of the pressure after or distal of a stenosis (e.g., P_{d}) relative to the pressure before the stenosis and/or the aortic pressure (e.g., Pₐ). In other words, FFR may be understood as P_{d}/Pₐ.

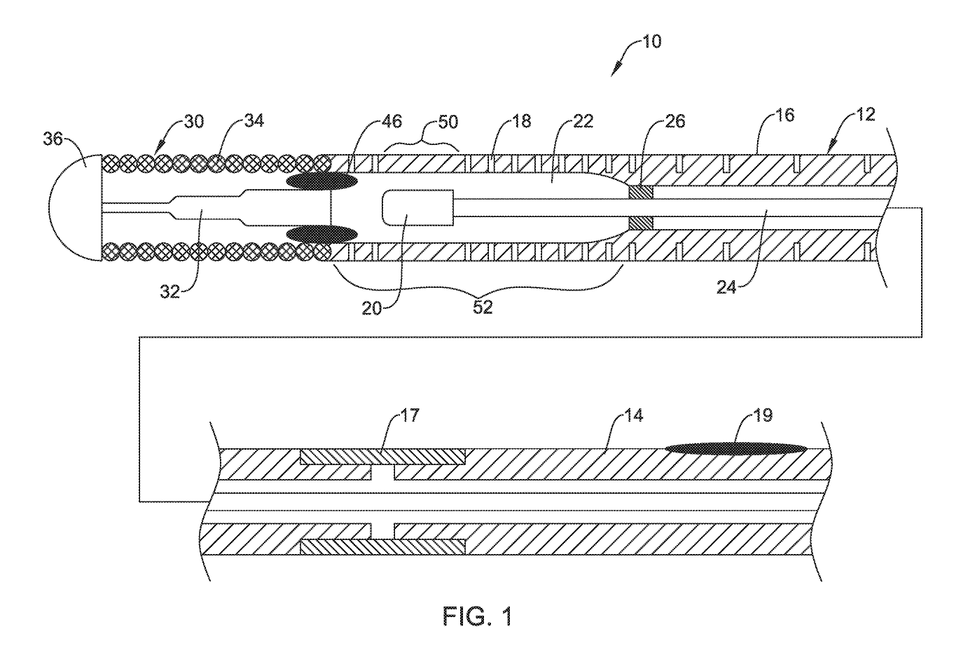

FIG. 1 illustrates a portion of an example medical device 10. In this example, medical device 10 is a blood pressure sensing guidewire 10. However, this is not intended to be limiting as other medical devices are contemplated including, for example, catheters, shafts, leads, wires, or the like. Guidewire 10 may include a tubular member or shaft 12. Shaft 12 may include a proximal portion 14 and a distal portion 16. The materials for proximal portion 14 and distal portion 16 may vary and may include those materials disclosed herein. For example, distal portion 16 may include a nickel-cobalt-chromium-molybdenum alloy (e.g., MP35-N). Proximal portion 14 may include stainless steel. These are just examples. Other materials may also be utilized.

In some embodiments, proximal portion 14 and distal portion 16 are formed from the same monolith of material. In other words, proximal portion 14 and distal portion 16 are portions of the same tube defining shaft 12. In other embodiments, proximal portion 14 and distal portion 16 are separate tubular members that are joined together. For example, a section of the outer surface of portions 14/16 may be removed and a sleeve 17 is may be disposed over the removed sections to join portions 14/16. Alternatively, sleeve 17 may be simply disposed over portions 14/16. Other bonds may also be used including welds, thermal bonds, adhesive bonds, or the like. If utilized, sleeve 17 used to join proximal portion 14 with distal portion 16 may include a material that desirably bonds with both proximal portion 14 and distal portion 16. For example, sleeve 17 may include a nickel-chromium-molybdenum alloy (e.g., INCONEL).

A plurality of slots 18 may be formed in tubular member 12. In at least some embodiments, slots 18 are formed in distal portion 16. In at least some embodiments, proximal portion 14 lacks slots 18. However, proximal portion 14 may include slots 18. Slots 18 may be desirable for a number of reasons. For example, slots 18 may provide a desirable level of flexibility to tubular member 12 (e.g., along distal portion 16) while also allowing suitable transmission of torque. Slots 18 may be arranged/distributed along distal portion 16 in a suitable manner including any of those arrangements disclosed herein. For example, slots 18 may be arranged as opposing pairs of slots 18 that are distributed along the length of distal portion 16. In some embodiments, adjacent pairs of slots 18 may have a substantially constant spacing relative to one another. Alternatively, the spacing between adjacent pairs may vary. For example, more distal regions of distal portion 16 may have a decreased spacing (and/or increased slot density), which may provide increased flexibility. In other embodiments, more distal regions of distal portion 16 may have an increased spacing (and/or decreased slot density). These are just examples. Other arrangements are contemplated.

A pressure sensor 20 may be disposed within tubular member 12 (e.g., within a lumen 22 of tubular member 12). While pressure sensor 20 is shown schematically in FIG. 1, it can be appreciated that the structural form and/or type of pressure sensor 20 may vary. For example, pressure sensor 20 may include a semiconductor (e.g., silicon wafer) pressure senor, piezoelectric pressure sensor, a fiber optic or optical pressure sensor, a Fabry-Perot type pressure sensor, an ultrasound transducer and/or ultrasound pressure sensor, a magnetic pressure sensor, a solid-state pressure sensor, or the like, or any other suitable pressure sensor.

As indicated above, pressure sensor 20 may include an optical pressure sensor. In at least some of these embodiments, a fiber optic cable 24 may be attached to pressure is sensor 20 and may extend proximally therefrom. An attachment member 26 may attach fiber optic cable 24 to tubular member 12. Attachment member 26 may be circumferentially disposed about and attached to optical fiber 24 and may be secured to the inner surface of tubular member 12 (e.g., distal portion 16). In at least some embodiments, attachment member 26 is proximally spaced from pressure sensor 20. Other arrangements are contemplated. In some instances, a centering ring (not shown) may be disposed around optical fiber 24 at a position that is spaced proximally from optical pressure sensor 20.

In at least some embodiments, distal portion 16 may include a region with a thinned wall and/or an increased inner diameter that defines a housing region 52. In general, housing region 52 is the region of distal portion 16 that ultimately “houses” the pressure sensor (e.g., pressure sensor 20). By virtue of having a portion of the inner wall of tubular member 12 being removed at housing region 52, additional space may be created or otherwise defined that can accommodate sensor 20.

In at least some embodiments, it may be desirable for pressure sensor 20 to have reduced exposure along its side surfaces to fluid pressure (e.g., from the blood). Accordingly, it may be desirable to position pressure sensor 20 along a landing region 50 defined along housing region 52. Landing region 50 may be substantially free of slots 18 so that the side surfaces of pressure sensor 20 have a reduced likelihood of being deformed due to fluid pressures at these locations. Distal of landing region 50, housing region 52 may include slots 18 that provide fluid access to pressure sensor 20.

Moreover, slots 18 may define a fluid pathway that allows blood (and/or a body fluid) to flow from a position along the exterior or outer surface of guidewire 10 (and/or tubular member 12), through slots 18, and into the lumen 22 of tubular member 12, where the blood can come into contact with pressure sensor 20. Because of this, no additional side openings/holes (e.g., other than slots 18) may be necessary in tubular member 12 for pressure measurement. This may also allow the length of distal portion 16 to be shorter than typical sensor mounts or hypotubes that would need to have a length sufficient for a suitable opening/hole (e.g., a suitable “large” opening/hole) to be formed therein that provides fluid access to sensor 20.

A tip member 30 may be coupled to distal portion 16. Tip member 30 may include a shaping member 32 and a spring or coil member 34. A distal tip 36 may be attached to is shaping member 32 and/or spring 34. In at least some embodiments, distal tip 36 may take the form of a solder ball tip. Tip member 30 may be joined to distal portion 16 of tubular member 12 with a bonding member 46 such as a weld.

Tubular member 12 may include a hydrophilic coating 19. In some embodiments, hydrophilic coating 19 may extend along substantially the full length of tubular member 12. In other embodiments, one or more discrete sections of tubular member 12 may include hydrophilic coating 19.

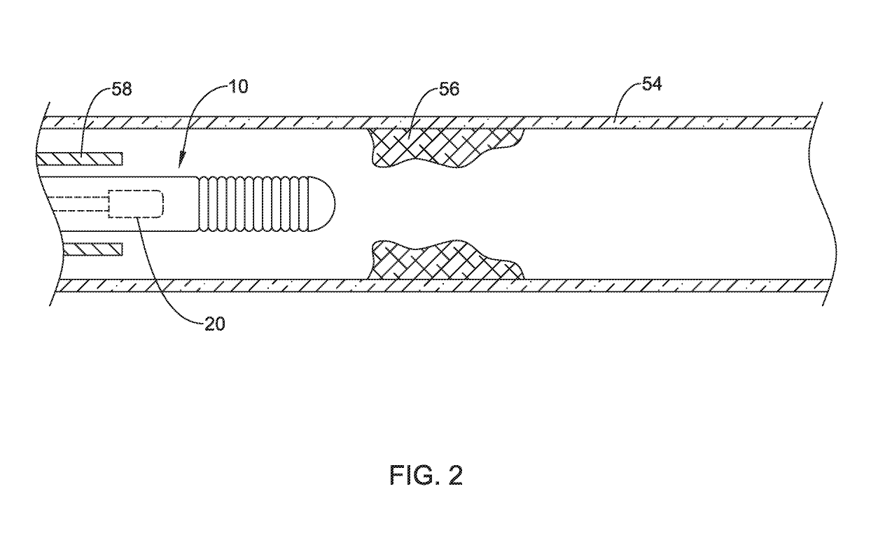

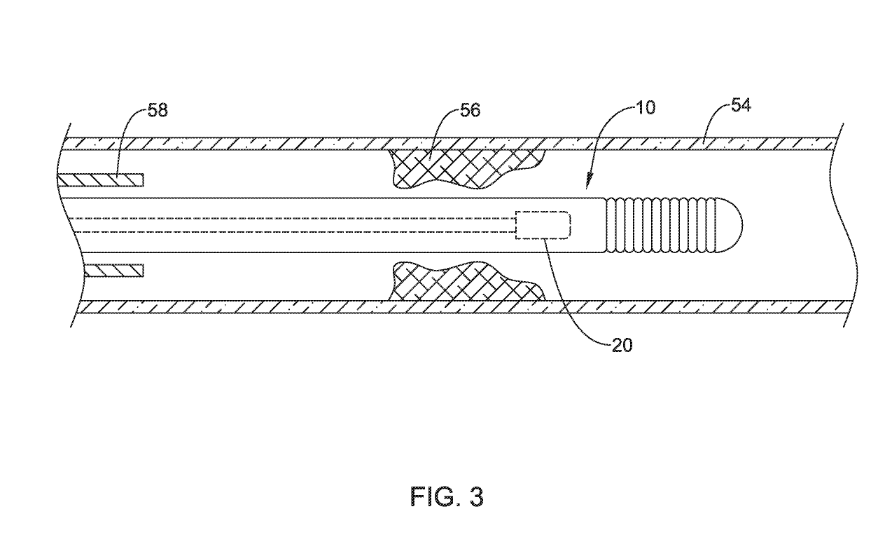

In use, a clinician may use guidewire 10 to measure and/or calculate FFR (e.g., the pressure after an intravascular occlusion relative to the pressure before the occlusion and/or the aortic pressure). Measuring and/or calculating FFR may include measuring the aortic pressure in a patient. This may include advancing guidewire 10 through a blood vessel or body lumen 54 to a position that is proximal or upstream of an occlusion 56 as shown in FIG. 2. For example, guidewire 10 may be advanced through a guide catheter 58 to a position where at least a portion of sensor 20 is disposed distal of the distal end of guide catheter 58 and measuring the pressure within body lumen 54. This pressure may be characterized as an initial pressure. In some embodiments, the aortic pressure may also be measured by another device (e.g., a pressure sensing guidewire, catheter, or the like). The initial pressure may be equalized with the aortic pressure. For example, the initial pressure measured by guidewire 10 may be set to be the same as the measured aortic pressure. Guidewire 10 may be further advanced to a position distal or downstream of occlusion 56 as shown in FIG. 3 and the pressure within body lumen 54 may be measured. This pressure may be characterized as the downstream or distal pressure. The distal pressure and the aortic pressure may be used to calculate FFR.

It can be appreciated that an FFR system that utilizes an optical pressure sensor in a pressure sensing guidewire may be connected to a number of processing/conditioning units, displays, and the like. When making these connections, the various cables/connections may be designed so that the optical signals can be transmitted between adjacent optical fibers in an efficient manner.

A wide variety of optical connectors exist that are designed to allow for efficient communication between adjacent optical fibers. Such connectors are typically utilized in industries such as telecommunication. The use of optical fibers in medical devices is provides a variety of new challenges. When optical fibers are utilized in medical devices, the connectors may need to allow for the connection of various devices and/or components while allowing for movement (e.g., rotation) of the components relative to one another during use. These movements could lead to complications. For example, the polished end surfaces of the fiber could contact one another, which could ultimately scratch, rub, or damage the fibers. This could impact the optical communication between the fibers. At least some of the medical devices, medical device systems, and connectors disclosed herein may include features that improve the connection of components of a fiber optic system such as the connection of optical fibers.

For the purposes of this disclosure, reference will be made to “medical device systems”. The medical device systems may be understood to be one or more medical devices that may be used together. In at least some embodiments, the medical device systems disclosed herein may be systems for measuring FFR. These systems may include a pressure sensing guidewire, an optical connector cable coupled to the guidewire, a signal conditioning unit and/or processing unit coupled to the optical connector cable, and a display unit or output. The systems may also include additional intermediate cables and/or devices, guide catheters, other pressure measuring devices and/or components, and the like. References made to a system are not meant to imply that all of these components are present.

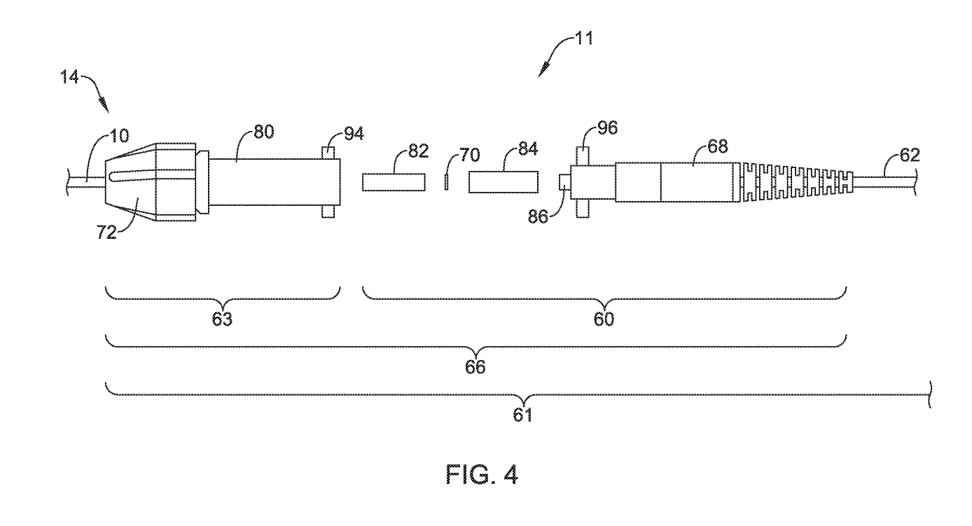

FIG. 4 schematically illustrates a portion of an example medical device system 11. For the purposes of the disclosure, system 11 may include pressure sensing guidewire 10 and an optical connector cable and/or cable assembly 61. In some instances, system 11 may include other components including, for example, a signal conditioning unit, other cables/connectors, display and/or processing components, and the like.

Connector cable assembly 61 may include a distal connector 66 and a cable body 62 extending proximally from distal connector 66. In at least some instances, distal connector 66 includes two releasably attachable components or portions. For example, distal connector 66 may include a first portion 60 and a second portion 63. Second portion 63 may be designed to be secured to guidewire 10. For example, second portion 63 may include a locking structure for releasably locking guidewire 10 to distal connector 66. In some instances, second portion 63 may resemble a torqueing device (e.g., a torqueing is device having a locking collet and a grasping region that allows a clinician to grasp and rotate the torqueing device). First portion 60 may include structural features that allow connector cable 61 to communicate with other components.

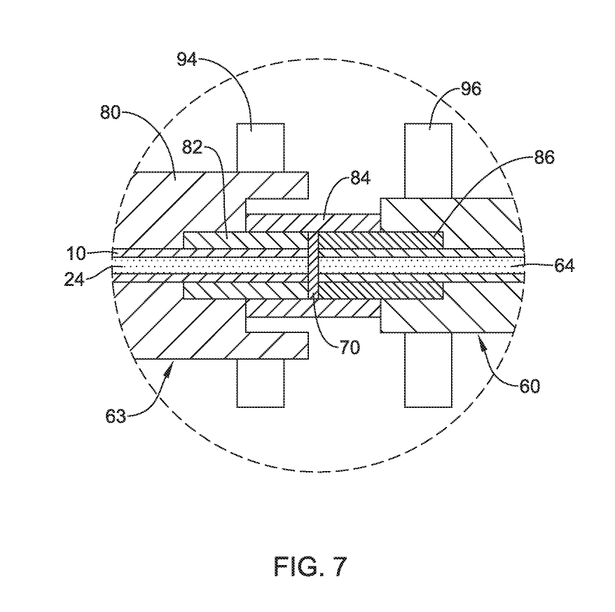

Second portion 63 may include body portion 80 and a rotatable member 72. In at least some instances, rotatable member 72 may be the nut of a collet assembly. Although not explicitly shown, rotatable member 72 may threadably engage body portion 80 (or another section of second portion 63) in order to secure guidewire 10 to second portion 63. First portion 60 may include a cable body 62, an optical fiber within the cable body (not shown in FIG. 4; shown schematically in FIG. 7), a ferrule 86, a split sleeve 84, a ferrule 82, optionally a spring (not shown), and optionally a flanged body (not shown) that is secured (e.g., via an adhesive bond or other suitable bond) to ferrule 86. Connector cable assembly 61 may be utilized to optically connect optical fiber 24 with fiber 64, which extends to one or more components of system 11 (and/or other systems) including, for example, a signal conditioning unit.

Movement and/or contact between adjacent optical fibers such as fibers 24/64 could lead to damage of the polished ends of the fibers 24/64. This could impact the communication between fibers 24/64. In order to improve the communication between fibers 24/64, a coupler 70 may be disposed within distal connector 66. Coupler 70 may be disposed between the ends of fibers 24/64. In at least some embodiments, coupler 70 may be a deformable disc or cylinder. For example, coupler 70 may take the form of a polymer disc. This may include a disc or cylinder formed from a compliant material such as an optically clear (e.g., aliphatic) polyurethane. Other forms are also contemplated for coupler 70. For example, coupler 70 may be a gel (e.g., a relatively thick gel), a coating on one or both of fibers 26/64, a membrane, or the like. Coupler 70 may be formed from one or more polymers or from other suitable materials including those disclosed herein. In at least some embodiments, coupler 70 may function as a “cushioning member” or a structural feature that provides some level of deformability at the interface between fibers 24/64 when bringing together fibers 24/64 (and/or bringing together guidewire 10 and optical connector cable 61).

In can be appreciated that optical fibers 24/64 may include an inner core and an outer cladding. In some instances, optical fibers 24/64 may have cores with the same is diameter (e.g., about 62.5 μm). In other instances, optical fibers 24/64 may have cores with differing diameters. For example, optical fiber 24 may have a core diameter of about 62.5 μm and optical fiber 64 may have a core diameter of about 105 μm. These are just examples. Other diameters are contemplated. In addition, the outer diameter of optical fibers 24/64 may be the same or different. For example, the outer diameter of optical fibers 24/64 may be about 125 μm. These are just examples. Other diameters are contemplated.

In at least some instances, second portion 63 and first portion 60 are designed to be coupled or otherwise connected to one another by a rotatable, magnetic connection. This may desirably aid in ultimately connecting guidewire 10 to cable 62 in a manner that is relatively simple and easy to accomplish. Furthermore, the connection may reliably secure guidewire 10 to cable 62 in a manner that still permits the rotation of guidewire 10. The magnetic connection may be accomplished using a plurality of magnets. For example, second portion 63 may include a first magnet 94 and first portion 60 may include a second magnet 96. Bringing magnet 94 into the proximity of magnet 96 may reliably hold together second portion 63 and first portion 60.

Magnetic coupling of the first portion 60 and the second portion 63 allows for a quick disconnect and reconnect at the magnets 94, 96. This allows for ease of handling for a quick disconnect and reconnect between the guidewire 10 and the collet 78.

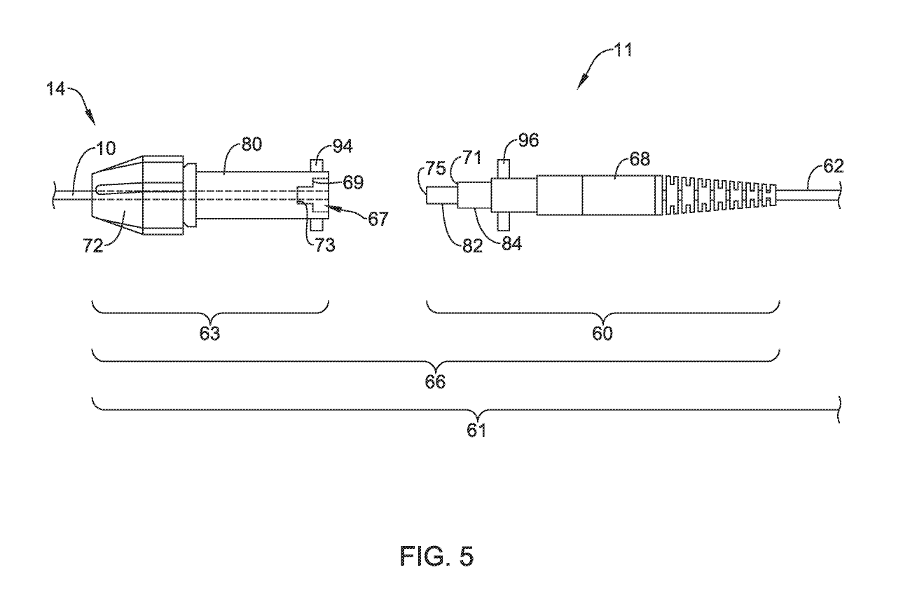

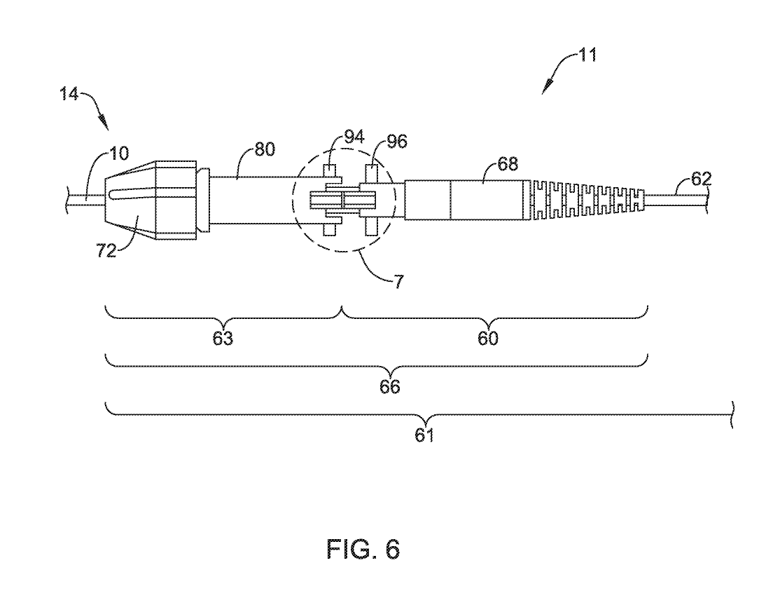

Magnetically coupling second portion 66 and first portion 60 is schematically illustrated in FIG. 5-7. In FIG. 5, second portion is shown assembled. Here it can be seen that split sleeve 84 may project out from the distal end of body 68. Ferrule 82 may project out from split sleeve 84. It can also be seen that body 80 may include a cavity or pocket 67 for receiving the distal end of first portion 60. More particularly, body 80 may include pocket 67 including a first shoulder region 69 designed to abut with a distal end 71 of split sleeve 84 and a second shoulder 73 that is designed to abut with a distal end 75 of ferrule 82. The configuration of pocket 67 may help to properly align second portion 63 with first portion 60 when coupling together portions 63/60 (e.g., as shown in FIGS. 6-7). In some instances, second portion 63 may not include pocket 67 or may include one or more structural variations on pocket 67.

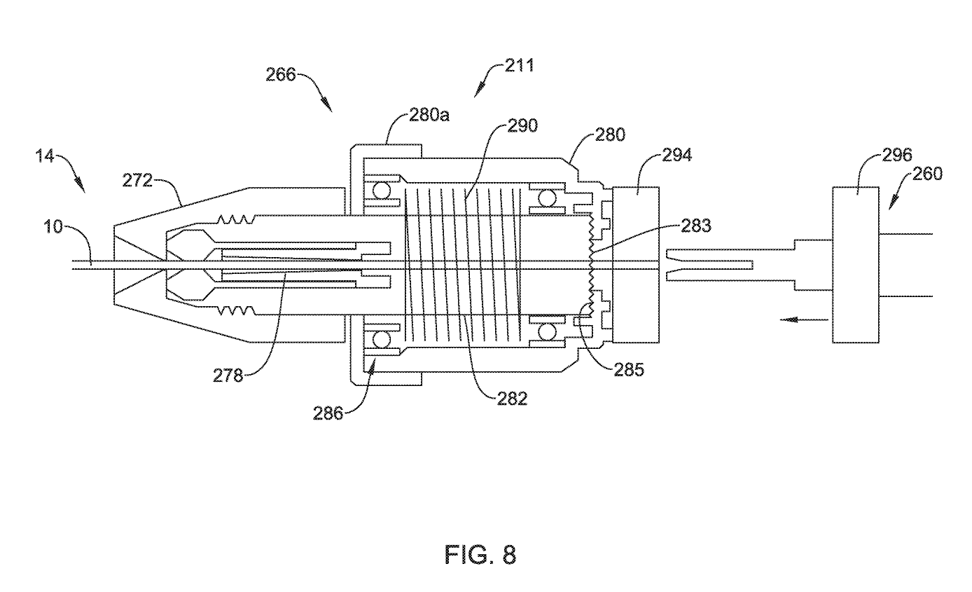

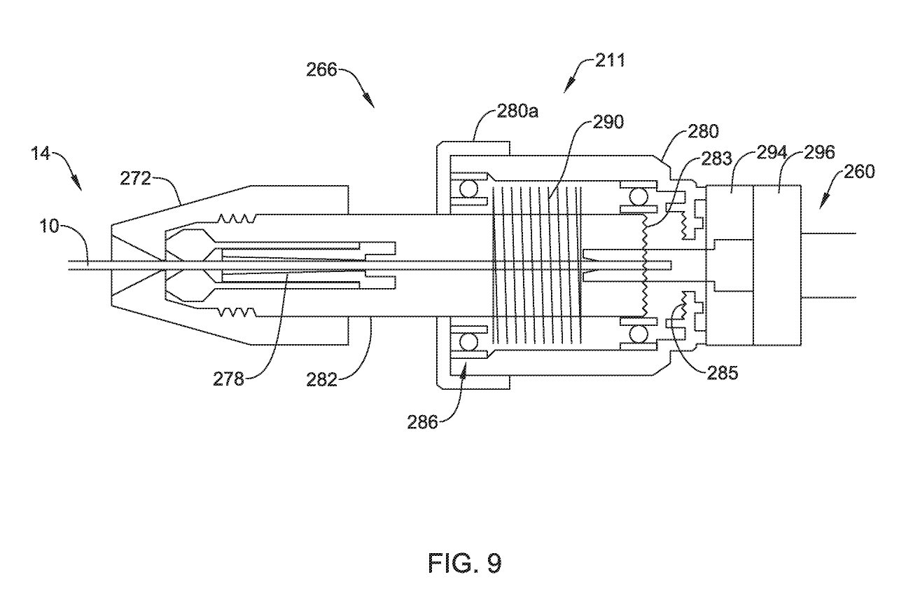

FIGS. 8 and 9 schematically illustrate a portion of example medical device system 211 including a pressure sensing guidewire 10 and a connector 266 that may be similar in form and function to other systems disclosed herein. FIG. 8 illustrates the medical device system 211 prior to connection of the distal connector 266 to the optical cable body 260 and FIG. 9 illustrates the device after connection of the distal connector 266 to the optical cable body 260.

Distal connector 266 may include body portion 280/280a, an inner assembly 282, a rotatable member 272, a collet 278, a spring 290, a distal bearing assembly 286, and a magnet 294. Rotatable member 272 may take the form of a collet nut or cap that can be rotated in order to tighten collet 278 onto guidewire 10. For example, guidewire 10 may be loaded into the distal connector 266 until it is flush with magnet 294. Collet cap 272 may be tightened in order to lock collet 278 to the guidewire 10 as shown in FIG. 8.

Magnetic coupling of the optical connector cable 260 and the distal connector 266 allows for a quick disconnect and reconnect at the magnets 294, 296. This allows for ease of handling for a quick disconnect and reconnect between the guidewire 10 and the collet 278.

Distal connector 266 (and/or the proximal portion 14 of guidewire 10) may be coupled to an example optical connector cable 260 shown in FIGS. 8 and 9 via magnetic attraction. Connector cable 260 may include an optical fiber within the cable body (not shown), a magnet or magnetic receptive material 296, and a split sleeve (not shown). Optical connector cable 260 may be utilized to optically connect optical fiber 24 with fiber 64, which extends to one or more components of system 211 including, for example, a signal conditioning unit.

Distal connector 266 may be a rotatable connector that allows guidewire 10 to be rotated relative to optical connector cable 260, for example at or otherwise about the bearing assembly 286. Prior to optical connector cable 260 being engaged with distal connector 266, spring 290 pushes against inner assembly 282 (in this example, spring 290 pushes inner assembly 282 in a proximal direction) in order to engage teeth 285 disposed along the proximal end of inner assembly 282 with corresponding teeth or grooves 283 along housing 280. When teeth 285 are engaged with grooves 283, distal connector 266 is substantially prevented from rotating relative to optical connector cable 260. This allows collet nut 272 to be rotated in order to secure guidewire 10 to distal connector 266. When optical connector cable 260 is pushed into contact with distal connector 266, the distal end of optical connector cable 266 pushes against guidewire 10 and moves inner assembly 282. Spring 290 is compressed and teeth 285 move to separate from grooves 283, while magnet 296 of the optical connector cable 260 allows locking of the collet 278 onto the guidewire 10 by turning the collet cap 272, which allows rotation of the medical device 211 at the collet 278. When the magnet 296 of the optical cable connector 260 is pulled away from the connector 266 of the proximal portion 14, the spring 290 pushes the teeth 285 of the distal connector 266 into the locked position with the grooves 283 of the distal connector 66 which locks the assembly. This allows rotation of the collet cap to unlock the collet 278 relative to the guidewire. A benefit of this particular arrangement is that rotation occurs at the bearing assembly 286.

Optical connector cable 260 may be lined up with and inserted into distal connector 266 until it pushes on guidewire 10 compressing the spring 290 and moving the inner assembly 282 in a distal direction, moving it away from the locking teeth 285 as shown in FIG. 9. This allows rotation at the distal bearing assembly 286.

Pulling the distal connector 266 away from spring 290 pushes the inner assembly 282 back into the locking teeth 285, allowing rotation of the collet cap 272 to unlock collet 278 from the guidewire 10, allowing removal of the guidewire 10.

Again, magnetic coupling of the distal connector 266 and the optical connector cable 260 allows for a quick disconnect and reconnect at the magnets 294, 296. This allows for ease of handling for a quick disconnect and reconnect between the guidewire 10 and the collet 278.

It should be noted that one of the magnet or magnetic materials 294 or 296 can be a magnet, and the other a magnetic receptive material, or both can be a magnet but having opposite polarity.

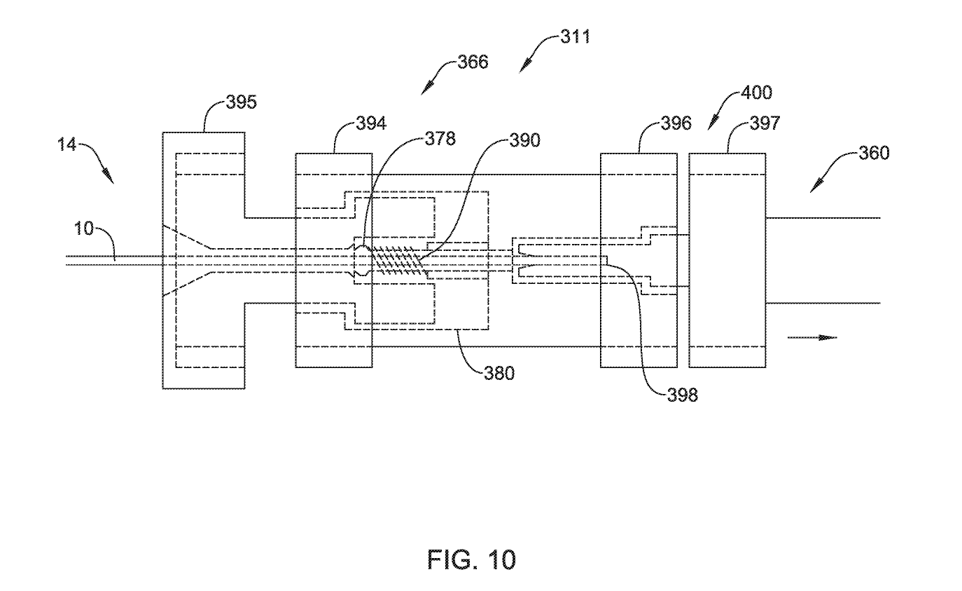

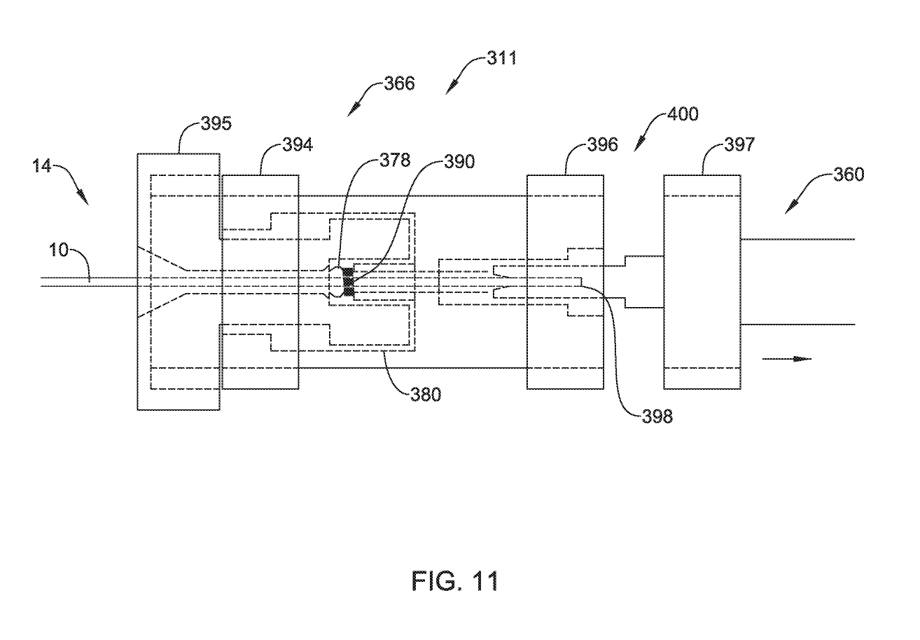

FIG. 10 schematically illustrates a portion of example medical device system 311 including a pressure sensing guidewire 10 and a connector 366 that may be similar in form and function to other systems disclosed herein. FIG. 9 represents proximal portion 14 of the guidewire 10 with connector 366 during coupling to an optical cable body 360.

Distal connector 366 may include a body portion 380, a spring 390 disposed within the body portion 380, a collet 378 and collet cap (not shown). In this embodiment, proximal portion 14 of guidewire includes two magnets 394, 396 of opposite polarity.

Here it can be seen that proximal portion 14 of guidewire 10 may be coupled to an example optical connector cable 360 via connector 366. Optical connector cable 360 may include an optical fiber within the cable body (not shown), and a split sleeve (not shown). Optical connector cable 360 may be utilized to optically connect optical fiber 24 with fiber 64 (not shown in this embodiment), which extends to one or more components of system 311 including, for example, a signal conditioning unit. In this embodiment, optical connector cable also includes two magnets 396, 397 of opposite polarity. It should be noted that the arrangement is such that magnets 395, 396 are of the same polarity, and magnets 394, 397 are of the same polarity but have a different polarity than magnets 395, 396.

It should also be noted that the arrangement may also include a magnetic receptive material 395, a magnet 394 of a first polarity, a magnet 396 of an opposite polarity to that of magnet 394, and a magnetic receptive material 397. Other arrangements are also within the purview of those of ordinary skill in the art providing that magnetic attraction result between 395 and 394, between 394 and 396, and between 396 and 397.

In this embodiment, the medical device system 311, rotates at the proximal magnetic arrangement 400.

When the guidewire 10 is inserted into distal connector 366, it stops at the proximal portion 398 of the distal connector 366. When magnet 395 is pushed towards magnet 394, magnet 394 is pushed into contact with spring 390, compressing the spring 390. Compression of the spring 390 compresses the collet 378 onto the guidewire 10. Once the collet 378 is locked onto the guidewire 10, it pushes on the distal magnet arrangement 400, pushing magnet 397 away from magnet 396 as shown in FIG. 11, allowing rotation at this point.

Magnetic coupling of the distal connector 366 and the optical connector cable 360 allows for a quick disconnect and reconnect at the magnets 394, 396. This allows for ease of handling for a quick disconnect and reconnect between the guidewire 10 and the collet 378.

The materials that can be used for the various components of guidewire 10 (and/or other guidewires disclosed herein) and the various tubular members disclosed herein may include those commonly associated with medical devices. For simplicity purposes, the following discussion makes reference to tubular member 12 and other components of guidewire 10. However, this is not intended to limit the devices and methods described herein, as the discussion may be applied to other similar tubular members and/or components of tubular members or devices disclosed herein.

The various components of the devices/systems disclosed herein may include a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available is from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

U.S. Patent Application Publication No. U.S. 2014/0350414 is herein incorporated by reference. U.S. Patent Application Publication No. U.S. 2014/0058275 is herein incorporated by reference. U.S. patent application Ser. No. 14/196,740 filed Mar. 4, 2014 is herein incorporated by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An optical connector cable assembly, the optical connector cable assembly comprising:
a first portion and a second portion, wherein the first portion is magnetically couplable to the second portion;
a first optical fiber is included with the optical connector cable assembly extending from the first portion; and
the second portion is configured to connect to a pressure sensing guidewire including an optical pressure sensor and a second optical fiber extending proximally from the optical pressure sensor.

2. The optical connector cable assembly of claim 1, wherein the first portion and the second portion are rotatably and magnetically couplable.

3. The optical connector cable assembly of claim 1, wherein the second portion is coupled to a rotatable connector.

4. The optical connector cable assembly of claim 1, wherein the rotatable connector is a rotatable collet.

5. The optical connector cable assembly of claim 1, wherein the optical connector cable assembly is configured to connect to a signal processing module.

6. The optical connector cable assembly of claim 2, wherein a magnetic force rotatably holds the first connector cable and the second connector cable together via a magnet and a magnetic receptive material.

7. The optical connector cable assembly of claim 1, wherein the first portion comprises a female adapter and the second portion comprises a male adapter.

8. The optical connector cable assembly of claim 7, wherein the female adapter is coupled to the pressure sensing guidewire.

9. The optical connector cable assembly of claim 1 wherein the first portion comprises a first magnetic having a first polarity and the second portion comprises a second magnet having a second polarity opposite to that of the first polarity.

10. A medical device system for measuring blood pressure, the system comprising:
an optical connector cable assembly including a first portion and a second portion magnetically couplable to the first portion, the first portion comprising an optical connector cable comprising a first optical fiber;
a pressure sensing guidewire including a pressure sensor and a second optical fiber extending proximally from the pressure sensor, the first optical fiber is cable of optically communicating the second optical fiber; and
wherein the second portion of the optical connector cable assembly is coupled to the guidewire.

11. The medical device system of claim 10 wherein the first portion and the second portion are rotatably and magnetically couplable.

12. The medical device system of claim 10 wherein the second portion includes a rotatable proximal connector capable of being rotatably coupled to the guidewire.

13. The medical device system of claim 10 wherein the rotatable proximal connector is a rotatable collet.

14. The medical device system of claim 10 wherein the pressure sensing guidewire comprises a tubular member having a proximal region and a distal region, the optical fiber is disposed within the distal region of the tubular, the optical fiber is coupled to the optical pressure sensor, the optical pressure sensor is disposed within the tubular member along the distal region.

15. The medical device system of claim 14 wherein a centering member is coupled to an outer surface of the first optical fiber and positioned adjacent to the optical pressure sensor, the centering member configured to reduce contact between an inner surface of the tubular member and the optical pressure sensor.

16. The medical device system of claim 14, wherein a centering member is coupled to an inner surface of the distal region of the tubular member.

17. The medical device system of claim 10, wherein the optical connector cable assembly is configured to connect to a signal processing module.

18. The medical device system of claim 10, wherein the first portion comprises a first magnet having a first polarity and the second portion comprises a second magnet having a second polarity opposite to that of the first polarity.

19. A method of connecting a pressure sensing guidewire to an optical connector cable, the method comprising:
advancing a first portion of an optical connector cable assembly into contact with a second portion of the optical connector cable assembly such that the first portion and second portion are magnetically held together and a first optical fiber of the optical connector cable assembly and a second optical fiber are in optical communication wherein,
the first optical fiber included with the optical cable assembly and the second optical fiber extends from a pressure sensing guidewire having a pressure sensor, the pressure sensing guidewire is coupled to the second portion of the optical connector cable assembly.

20. The method of claim 19 wherein the first portion and the second portion are rotatably and magnetically coupled.
